(19) United States
(12) Patent Application Publication
(10) Pub. No.: US 2018/0372742 A1
(43) Pub. Date: Dec. 27, 2018

(54) Babesia Biomarkers for Diagnostic and Screening In Vitro Diagnostic Test

(71) Applicant: ImmPORT Therapeutics, Inc., Irvine, CA (US)

(72) Inventors: Douglas Molina, Buena Park, CA (US); Arlo Randall, Huntington Beach (US); Gary Hermanson, Encinitas, CA (US); Choukri Ben Mamoun, Irvine, CA (US)

(21) Appl. No.: 16/060,381

(22) PCT Filed: Dec. 9, 2016
(86) PCT No.: PCT/US2016/065862

§ 371 Date: Jun. 7, 2018

Related U.S. Application Data
(60) Provisional application No. 62/265,947, filed on Dec. 10, 2015.

(51) Int. Cl. G01N 33/569; G01N 33/543

(52) U.S. Cl. CPC G01N 33/56905; G01N 33/54306; G01N 2800/26; G01N 2469/20; G01N 2333/44

(57) ABSTRACT

An antigen composition with plurality of antigens from B. microti, and methods of predicting a likelihood of an individual having B. microti infection using the antigen composition as biomarkers of infections of B. microti to human are provided. In particularly preferred aspects, the antigens are immunodominant and have quantified and known relative reactivities with respect to sera of a population infected with the pathogen with an average discriminatory p value of ≤0.05.

This application claims the benefit of priority to U.S. Provisional Patent Application No. 62/265,947, filed Dec. 10, 2015, which is incorporated by reference herein in its entirety.

### FIELD OF THE INVENTION

The field of the invention is identification of biomarkers for screening Babesia microti infection using an in vitro diagnostic test.

### BACKGROUND

The background description includes information that may be useful in understanding the present invention. It is not an admission that any of the information provided herein is prior art or relevant to the presently claimed invention, or that any publication specifically or implicitly referenced is prior art.

Human babesiosis is a multisystem disease endemic in the United States with a clinical presentation ranging from asymptomatic infection, or mild febrile illness, to a rapidly fatal disease. The mortality rate is about 9% in healthy individuals and 28% in inurame-compromised patients. The disease is caused primarily by Babesia microti (B. microti), an obligate intraerythrocytic apicomplexan parasite transmitted to humans by the tick vector, Ixodes scapularis, the same tick that transmits Borrelia Burgdorferi, the agent of Lyme disease, B. microti can also be transmitted through transfusion of blood from Babesia-infected asymptomatic donors. Babesiosis is a major public health concern and has become as a nationally notifiable disease following CDC recommendation in 2011. Diagnosis of symptomatic cases is often difficult because B. microti infection can be asymptomatic in immunocompetent individuals. Thus, the percentage of the population with current or past infection is largely underestimated.

Currently, no available tests are sensitive and specific enough to be suitable for B. Microti infection test in human blood. For example, indirect innimnofluorescence assay is the most widely used test for detection of circulating B. microti antibodies in human blood. However, the assay is laborious, not sufficiently sensitive and not amenable to large-scale epidemiological surveys or blood screenings. For another example, blood smear detection using light microscopy remains the best method in practice for clinical diagnosis, similar to malaria diagnosis. However, this method requires experienced microscopists for identification of intraerythrocytic parasites, which is labor intensive. Further, this method also suffers from lack, of sensitivity and specificity. Indeed, no FDA-licensed serological, molecular method or other type of diagnostic test is available for screening the blood supply. The current FDA-recommended approach for Transfusion Transmitted Babesiosis (TTB) prevention relies on the Uniform Donor Health History Questionnaire self-reporting method to identify individuals having a history of Babesiosis.

Screening for IgG antibodies against immunodominant recombinant B. microti antigens, such as secreted antigen 1 or the BMN family showed a possibility that those antigens may be prevalent among B. microti infected organisms. However, there are few studies in humans and even those human studies are usually limited by both sample size and the number of proteins tested. In addition, genomic data and annotation to predict high value targets has not been available. Thus, it has been essentially impossible to systematically categorize protein families and prioritize them in silico, and to test all members of categories that showed potential diagnostic utility.

In addition, a rapid, reliable screening method remains elusive because of low level parasitemia that can be variably and intermittently detected by parasitological or molecular methods. Furthermore, due the sampling conundrum, even highly sensitive molecular assays such as PCR cannot guarantee that a pint of blood is free of B. microti-infected erythrocytes, since only a small (˜1 ml) sample can be examined to assess the integrity of any given blood donation. Thus, a combination of rationally-designed molecular and serological methods is needed. However, current serological detection which relies on detection of IgG antibodies is complicated by the seronegative “window.” This is primarily in the early stages of infection, before individuals seroconvert and an IgG antibody response is developed.

Therefore, there is still a need for improved detection methods and identification of biomarkers for B. microti infected blood.

### SUMMARY OF INVENTIONS

In addressing the need for improved compositions and methods of antigen and antibody detection and monitoring for diagnostic and therapeutic applications related to B. microti. infection, the instant inventors utilized a proteome-microarray approach to profile the antibody response during infection against multiple B. microti proteins in order to identify antigens that generate antibodies with high avidity and specificity that may have more precise serodiagnostic and therapeutic utility.

Proteome microarrays were produced that displayed each of the proteins encoded by B. microti as expressed in E. coli-based in vitro transcription/translation of open reading frames (ORFs). Microarrays thus produced were screened with sera from a panel of patients with diagnosed with B. microti infection. A multiplex proteomics approach to characterize infected individual's antibody response to B. microti was discovered in which highly sensitive antigens were used in conjunction with highly specific antigens. To the inventors' knowledge, such a sensitive and specific characterization of the B. microti infection has not been previously attempted and/or accomplished.

The instant inventive subject matter provides a new and useful tool that can accurately survey B. microti infection and its onset and progress. More specifically, the present inventive subject matter provides tools, methods, and compositions for the identification, analysis, and monitoring of specific B. microti antigens, or sets of antigens, that have diagnostic, prognostic, and therapeutic value, specifically with respect to diagnosis and treatment of the human diseases or symptoms related to B. microti infection. Further, this instant inventive subject matter can provide clear, distinct, antigen targets for serodiagnostic, biomarker, vaccine, and therapeutic product development against B. microti infection and the diseases and disorders triggered by B. microti infection in humans. In addition the antigens may be expressed in E. coli, simplifying their production on a large scale.

The inventive subject matter provides an antigen composition for detecting and monitoring for B. microti infection. The antigen composition includes a plurality of antibody reactive antigens associated with a carrier, of which at least two of the antigens have quantified and known relative antibody reactivities with respect to sera of a population infected by B. microti. It is preferred that one of the pluralities of antigens are selected from the group of antigens that are highly discriminatory either in the tests of IgG reactivity or IgM reactivity or both.

Another inventive subject matter provides a test kit for testing Babesia microti infection in patients suspected to have Babesia microti infection. The test kit includes a plurality of antibody reactive antigens associated with a carrier, and at least two of the antigens have quantified and known relative antibody reactivities with respect to sera of a population infected by Babesia microti.

Still another inventive subject matter provides a use of a plurality of antibody reactive antigens associated with a carrier in diagnosing Babesia microti infection. The plurality of antibody reactive antigens are preferably associated with a carrier, and at least two of the antigens have quantified and known relative antibody reactivities with respect to sera of a population infected by Babesia microti.

In a preferred embodiment, the antigens have an average discriminatory p value of ≤0.05. It is contemplated that those antigens include at least, but not limited to, those listed in Tables 1, and also shown in FIGS. 1-4. For example, such antigens includes BMG09; BMN1-9; BmSA1 (Gene ID: BBM _03g00785), rhoptry neck protein 2 (Gene ID: BBM_03g04695), hypothetical proteins (Gene ID: BBM_01g00985, Gene ID:BBM_03g00947, Gene ID: BBM_03g00960, BBM_04g06560, BBM_02g04285) Nucleoporin FG repeat region (Gene ID: BBM_03g02345) BMN family (2) (Gene ID: BBM_04g09980, DNA mismatch repair protein (Gene ID: BBM_03g03480, Actin (Gene ID: BBM_03g02390), and so on.

In some embodiments of the inventive subject matter, the carrier may be a pharmaceutically acceptable carrier suitable for use in a vaccine. In such an embodiment the vaccine may include four or more antigens and/or fragments thereof; such antigens or fragments thereof may be recombinant and/or at least partially purified. In another embodiment of the inventive subject matter, the carrier may be a solid and/or insoluble phase suitable for use in a diagnostic assay, such as, for example, an array or microarray, antigens and/or fragments thereof may be disposed upon such a carrier. In such an embodiment, the antigens or fragments thereof can be recombinant, and may be at a purity of at least 60% or least partially purified.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A shows an exemplary protein array of B. microti in human serum samples.

FIG. 1B show a heat map of a subset of B. microti proteins in unexposed and infected human IgG samples.

FIG. 1C show a heat map of a subset of B. microti proteins in unexposed and infected human IgM samples.

FIG. 2A show a heat map of another subset of B. microti proteins in unexposed and infected human IgG samples.

FIG. 2B show a scaled heat map of another subset of B. microti proteins of FIG. 2A in unexposed and infected human IgG samples.

FIG. 2C show a heat map of another subset of B. microti proteins in unexposed and infected human IgM samples.

FIG. 2D show a scaled heat map of another subset of B. microti proteins of FIG. 2C in unexposed and infected human IgM samples.

FIG. 3A shows an exemplary protein array of B. microti in infected and uninfected mouse serum samples.

FIG. 3B shows an exemplary heat map of B. microti in infected and uninfected mouse serum samples.

FIG. 4A an exemplary heat map and a bar graph of B. microti antigens reactive to IgG in field mice that was previously tested for B. Microti infection using the IFA gold standard test.

FIG. 4B an exemplary heat map and a bar graph of B. microti antigens reactive to IgM in field mice that was previously tested for B. Microti infection using the IFA gold standard test.

### DETAILED DESCRIPTION

In the present application, the inventors identified a group of antigens that shows relatively high reactivities to IgG and/or IgM of an infected population to B. microti. At least some of these antigens are highly specific and sensitive such that they are suitable to be used as biomarkers of B. microti infection.

The present inventive subject matter provides for the identification, analysis, and monitoring of antibody reactivity to specific B. microti antigens, or antigen sets, which has diagnostic, prognostic, and therapeutic value, specifically with respect to various diseases, disorders, and symptoms related to B. microti infection. The present inventive subject matter also provides tools and methods to accurately survey B. microti infections, disorders, and diseases via the combination of antigen/antibody reactivity detection and monitoring, and characterizing sera samples of uninfected and infected by B. microti.

It should be noted that in the following description antigens may be identified by either the gene descriptor for the gene that encodes the protein antigen and/or the name for the protein antigen. Thus, it should be understood that where the context indicates that a sequence or antigen is a protein sequence, a gene name for that sequence or antigen denotes the protein product for that gene. Where reference is made to antibodies it is recognized that while such antibodies may be referred to as having been obtained from serum, said antibodies may also derived from other sources, including, but not limited to, mucus, saliva, semen, lacrimal fluid, urine, feces, aqueous humor, pus, cerebrospinal fluid, lymphatic fluid, and synovial fluid.

The present inventive subject matter is also directed to the identification of specific B. microti antigens that trigger antibody reactivity associated with various diseases, disorders, or symptoms related to B. microti infection, which the specific antigens have predetermined antibody reactivities from serum of a population of B. microti Infected patients. Thus, such specific antigens may have a statistically high probability to elicit antibody responses in a relatively large group of B. microti infected patients.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Not withstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified, thus fulfilling the written description of all Markush groups used in the appended claims.

One aspect of the invention includes an antigen composition that is specific and sensitive to be a biomarker of B. microti infection. The antigen composition includes a plurality of antibody (IgG, IgM) reactive antigens. At least two of these antigens have quantified and known relative antibody reactivities with respect to sera of a population infected by Babesia microti. It is highly preferred that among these antigens, at least one antigen has an average discriminatory p value of ≤0.05.

The biomarker antigens of B. microti infection are identified from a proteome screen against sera of a population that has been previously exposed to the B. microti, which can be compared with sera of a population that has not been exposed to the B. microti. It is preferred that the screening also provides data of relative reactivities with respect to the antigens and sera of the populations

While not limiting to the inventive subject matter, in a preferred embodiment, the plurality of antigens are selected as a sampling of the B. microti secretome, GPI-anchored proteome, the metabolic proteome and other proteins. The secretome is the totality of secreted organic molecules and inorganic elements by biological cells, tissues, organs, and organisms. The GPI-anchored proteome is the totality of surface proteins anchored to the cell membrane via a GPI anchor. Because secreted proteins and surface proteins are naturally exposed to outside of the infecting organism (B. microti) and can be detected by antibodies in the infected organisms' sera, the GPI-anchored proteome and secretome are considered useful as antigens for diagnosing pathogen infections.

It is generally preferred that at least part of the B. microti's genome is obtained and all potential open reading frames and portions thereof are determined in silco. Once the potential genes are identified, suitable primers are determined to provide amplicons of the entire Open Reading Frames (ORFs), or, less preferably, portions thereof, when in the primers are preferably designed to allow facile subcloning into an expression system. Most preferably, the subcloning uses recombinase-based subcloning using unpurified PCR mixtures to avoid cloning bias, and the so obtained recombinant plasmids are polyclonally multiplied, which enables unbiased presentation of the amplicons. It is still further particularly preferred that the plasmid preparations are then subjected to an in vitro transcription/translation reaction to thereby provide the recombinant ORF peptide, which is then spotted or otherwise immobilized onto a suitable addressable carrier (e.g., membrane, bead, etc.).

It should be recognized that the so prepared proteomes can then be exposed to serum of a population of control individuals and/or population of individuals that are known to have exposure to B. microti from which the ORFs were prepared. Antibodies of the serum that bind to one or more of the ORFs are then detected using well known methods (e.g., use of secondary antibodies etc.). In this manner, the entire proteome of the pathogen can be rapidly assessed for immunogenicity and potential binding with antibodies in serum. Various preferred aspects, compositions, and methods of proteome preparation are disclosed in International patent publication number WO 06/088492, which is incorporated by reference herein.

It should be also noted that not all individuals show same response or development of antibodies to an antigen when they are exposed (or infected) by the antigen. Such individual differences in immune responses are, at least in part, due to their variability and specificity of major histocompatibility complex (MHC) types. Thus, an antigen that can be highly immunogenic to individual A may not be immunogenic to individual B. That being said, it is virtually impossible to predict whether a protein from a pathogen would be an immunogenic to human being by simply determining the types of the protein. Consequently, it is also virtually impossible to predict or determine an antigen or a set of antigens simply by looking at their protein sequences or genomic sequences whether such antigens would be immunogenic to human population. Therefore, and among various other advantages, it should be especially recognized that contemplated compositions and methods presented herein will allow for preparation of diagnostic compositions comprising a plurality of antigens with known and predetermined affinity to target ORFs of B. microti. As individual immune systems are known to exhibit significant variation with respect to antigen recognition, methods and compositions contemplated herein will allow statistically supported antigen identification to identify immunodominant antigens in a population of infected patient. Consequently, multiple targets can be used to elicit an immune response and/or detect a prior exposure, even where one or more of the targets may be evasive for detection or provide only a weak response.

With respect to the immunodominant sequences identified herein, it should be further appreciated that the sequences need not be complete ORFs, but that suitable sequences may also be partial sequences (e.g., synthetic, recombinant or isolated, etc.) that typically comprise at least part of an antigenic epitope. For example, contemplated DNA sequences include those that will hybridize under stringent hybridization conditions to respective sequences listed in the sequence listing. Thus, sequences contemplated herein may be identified as DNA sequences encoding the antigenic peptide (partial or entire ORF), or may be identified as peptide sequence (or homologs thereof). Similarly, chemically modified antigens, and/or orthologs of the polypeptides presented herein are also deemed suitable for use herein.

It should be particularly noted that while proteome screening will provide a plurality of antigens as potentially useful molecules for diagnosis, vaccination, and/or therapy, such an approach only provides a raw cut of (a plurality) of individual responses. Therefore, as most individual immune reactions towards the same pathogen elicit a significantly distinct profile of antibodies (e.g., depending on disease stage, previous exposure, and/or inter-individual variability), results obtained from such screening are typically inhomogeneous. Consequently, variability of the individual immune responses and variability of the quantity of recombinant protein in the array must be taken into consideration to obtain meaningful results.

Therefore, it should be appreciated that filtering of raw data will result in a collection of antigens with quantified and known relative reactivities with respect to sera of a population infected with B. microti. Moreover, it should be noted that as signals may be specific to a particular stage in the course of an infection, relative reactivities may be indicative of the time course of the infection, and/or relative reactivities may represent differences in the strength of immunogenicity of the particular antigen (or quantity of deposited antigen in the screening assay). Additionally, it should be particularly recognized that depending on the choice of the specific patient population, the tested sera will reflect the immune status of a population that is characterized by one or more parameters of the disease. For example, populations may be observed that are infected or not infected, that had a long-term exposure or chronic infection, which had spontaneous recovery, that represents a group of responders (or non-responders) to a particular drug treatment, or that had at least partial immunity to the pathogen.

In still further contemplated aspects, immunodominant antigens are identified by selecting for an antigen that (a) produces in at least 50% of a population a measurable signal, preferably in at least 60% of a population, and (b) has a signal strength of at least 40%, preferably at least 50% of the overall average signal intensity. However, and more preferably, the signal strength will be at least above average of the overall average signal intensity, and even more preferably in the upper tertile (quartile, or even quintile) of sizial intensities in the assay. Still further, it is generally preferred that the series of tests also include a negative control against which the potential immunodominant antigens are compared. Alternatively, immunodominant antigens can be identified by selecting for an antigen that shows high average discriminatory power and as such have a t-test p-value of ≤0.05, an area under the curve (AUC) of >0.75, and most preferably a Benjamini-Hochberg corrected p-value of ≤0.05.

Consequently, and with particular respect to the pathogen (B. microti) presented herein, it should be appreciated that compositions comprising one or more selected immunodominant antigens can be prepared that will have a statistically high probability to elicit or have elicited an immune response in a relatively large group of patients. Each of the antigens was characterized, inter alia, with regard to their individual and relative reactivities for the pathogen. Most typically, reactivity was measured as strength of immunogenicity (e.g., such that average binding affinity and/or average quantity of the antibodies produced a predetermined signal intensity (e.g., in the upper half, upper tertile, or even upper quartile)). In a preferred embodiment, the relative reactivities are quantified based on the antigens' reactivities to IgG. In some other embodiments, the relative reactivities are quantified based on the antigens' reactivities to IgM. In still some other embodiments, the relative reactivities are quantified based on the antigens' reactivities both to IgG and IgM. Further, it is contemplated that the relative reactivities of antigens suitable to be selected as biomarkers of B. microti infection are at least 30% higher, preferably at least 50%, compared to with respect to sera of a population uninfected by B. microti.

Preferably, the antigens used as biomarkers have high average discriminatory power and as such have a t-test p-value of ≤0.05, an area under the curve (AUC) of >0.75, and most preferably a Benjamini-Hochberg, corrected p-value of ≤0.05.

Selected antigens as biomarkers can be associated with a carrier. In some embodiments, carriers can be solid carriers (e.g., a plate, a membrane, a bead, a chip, a microfluidic device, etc.). In other embodiments, carriers can be non-solid carriers (e.g., liquid carriers, gas carriers, etc.). In the embodiments where the carriers are solid carriers, it is preferred that the antigens are deposited in an array. For example, antigens can be arrayed on a chip that can be read in an automated device (e.g., via scanner) or visual manner (e.g., dye-forming colorimetric reaction, etc.). Most typically, in these embodiments, the plurality of antigens is deposited in a spatially addressable manner (e.g., x-y matrix or beads with color association or microtiter plate, etc.). Moreover, it should be noted that diagnostic devices contemplated herein may be based on numerous well known manners of detection, including ELISA (sandwich or non-sandwich), competitive ELISA, anti-idiotypic antibodies, etc., wherein all known colorimetric and photometric (e.g., fluorescence, luminescence, etc.) or radiometric reactions are deemed suitable for use.

More specifically, in these embodiments, a plurality of immunodominant antigens of B. microti are deposited on a solid surface or onto an addressable solid phase and exposed to blood, serum, plasma or other antibody-containing body fluid. Consequently, so prepared compositions can be employed to identify and/or characterize an immune response of an individual against selected antigens, and optionally assess the kind of immune response (e.g., identification of latent or chronic infection, etc.), as well as disease progression, efficacy of therapy, etc. Most typically, the plurality of antigens will include between 5 to 10 antigens, but significantly higher amounts of antigens are also contemplated, including at least 25%, more typically at least 50%, even more typically at least 75%, and most typically at least 90% of the proteose of the pathogen. Similarly, less than 5 antigens (1-4) are also deemed suitable. In further typical aspects of the inventive subject matter, contemplated arrays are most preferably processed in a microfluidic device. For example, an array of antigens in such devices may be printed on a membrane or other material (e.g., nitrocellulose-coated carrier of less than 1 cm² area) that is then placed in a microfluidic device having sample/reagent inlet and outlet ports. Depending on the specific configuration, signals may be acquired using optical methods (e.g., charge coupled device (CCD) chip, flat bed scanner, etc.), electrical methods (e.g., voltametric or amperometric, etc.), or other methods well known in the art. Alternatively, visual detection or detection using a regular flat bed scanner at 1200 dpi resolution and/or fluorescence detection is also deemed suitable.

It is contemplated that antibodies against at least some immunodominant antigens may block invasion of B. microti to red blood cells. Thus, those immunodominant antigens can be effective therapeutic compositions or vaccines to B. microti infection. Thus, in another aspect of the inventive subject matter, the carrier is a pharmaceutically acceptable carrier, and the one or more immunodominant antigens are formulated as a vaccine or therapeutic compositions. In such aspects, it is generally preferred that the vaccine comprises multiple (e.g., at least two, four, or six, etc.) antigens. Depending on the antigens, it is contemplated that the antigens or fragments thereof are at least partially purified and/or recombinant.

### EXAMPLES

The inventors have identified biomarkers to include in a test that are detected by IgG and/or IgM antibodies to overcome the challenges of early detection of B. microti infection. Further, the inventors have used genomics and statistical analysis to clone high priority targets and tested them all for diagnostic utility. More importantly, the inventors identified at least 54 B. microti proteins that can be used alone or in combination to develop an in vitro diagnostic (IVD) for Babesiosis and a blood screening test for TTB.

In these examples, the inventors interrogated the targeted B. microti protein array with at least 34 human samples with known B. microti exposure, at least 60 wild mice with known immunofluorescence antibody assay (IFA) titers and at least 4 experimentally infected mice.

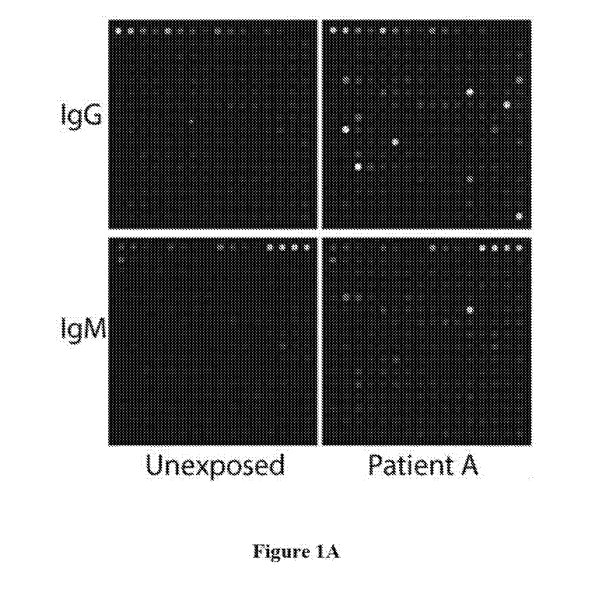

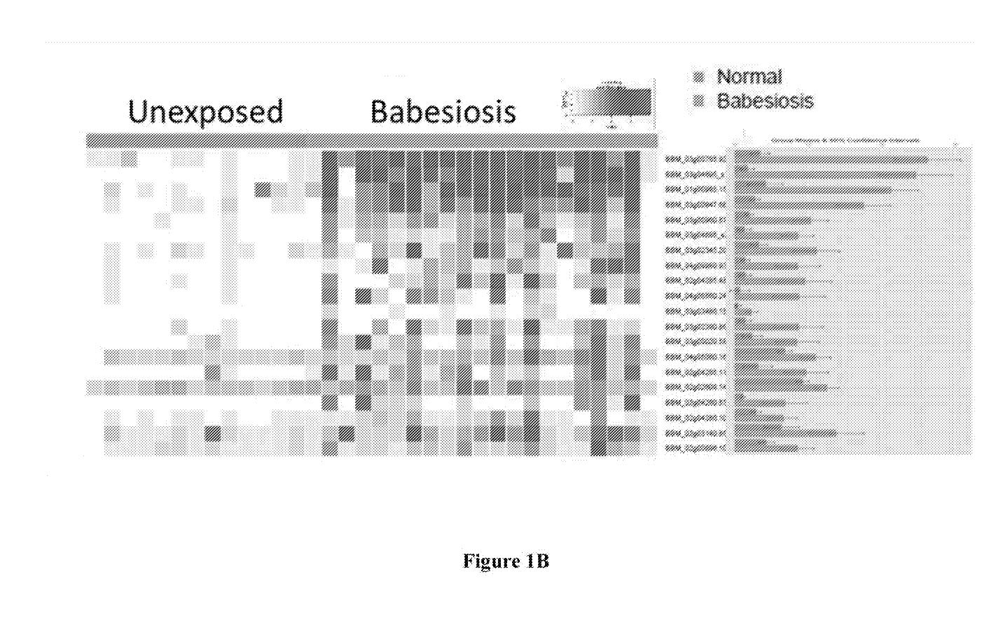

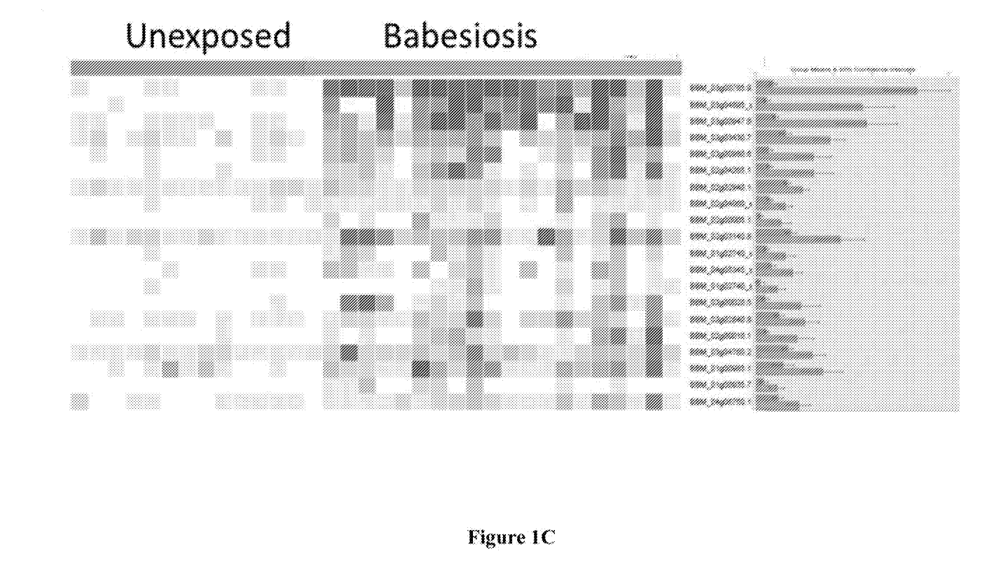

FIG. 1A shows an exemplary image of B. microti protein arrays used to detect IgG and IgM antibodies in normal serum of uninfected person and in serum of B. microti infected patient. Antibodies present in serum (or other bodily fluid including, but not limited to, mucus, urine, saliva, amniotic fluid, etc.) were detected using microarray technology. Bound antibodies were visualized using secondary antibodies conjugated to a fluorophore and scanned using a microarray scanner. Fluorescence intensity data were quantified and a data matrix is created where columns represent samples and rows represent proteins.

In FIGS. 1B and 1C, each figure shows 20 exemplary antigens selected based on the relative reactivities of antigens to IgG and IgM, respectively, represented as heat maps and bar graphs. The heat maps represent the intensity of antibody titer in the sample represented by a color scale (most intensive in dark grey, and least intensive in white). As shown in FIGS. 1A-C, uninfected humans and infected patients have very distinct antibody profiles. For example, test results of infected patients show significantly high intensities (shown in black in a heat map) of IgG or IgM titers in at least 5-7 antigens compared to uninfected samples (black colors), and showed relatively higher intensity of IgG or IgM titers in almost all rest of antigens tested (dark grey colors), while very few test results of uninfected show some IgG or IgM titers (shadow of grey colors), but no high intensity of antibody titers. These differences can be exploited to create a test to diagnose Babesiosis in individual samples. Such a test can also be further developed for large-scale screening of the blood supply for potentially infected blood to prevent TTB.

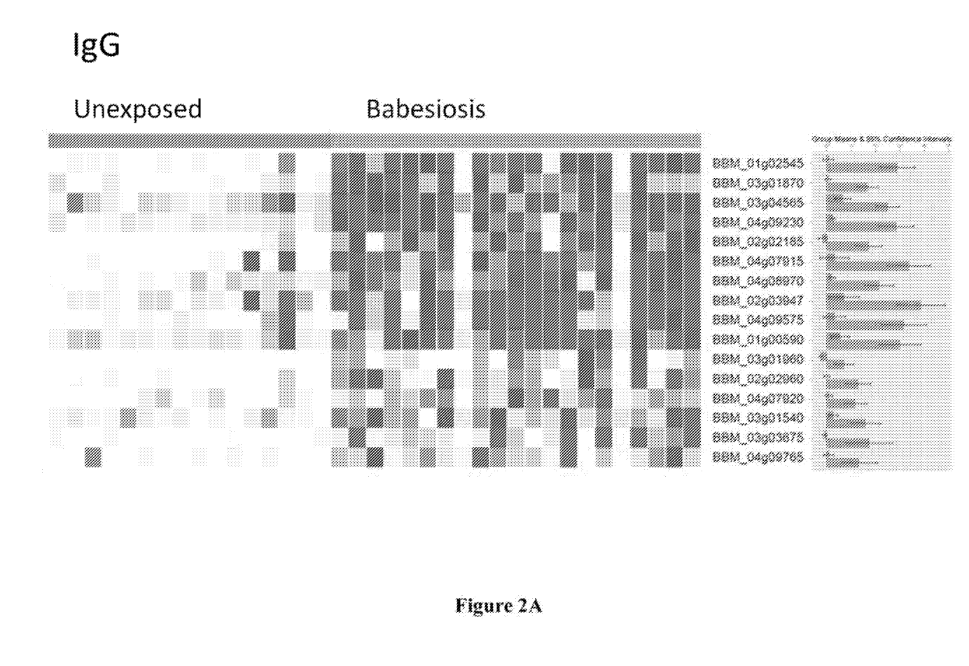

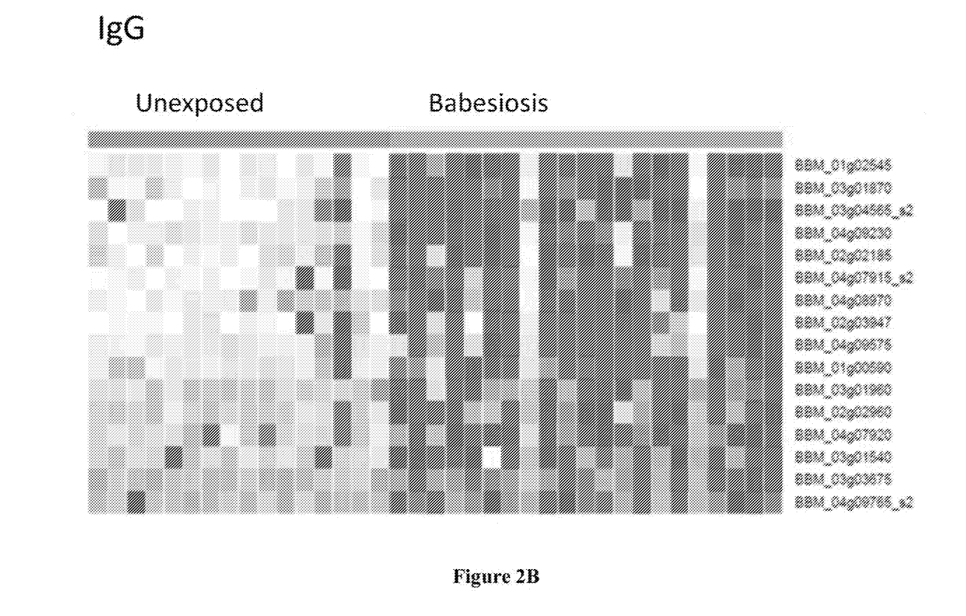

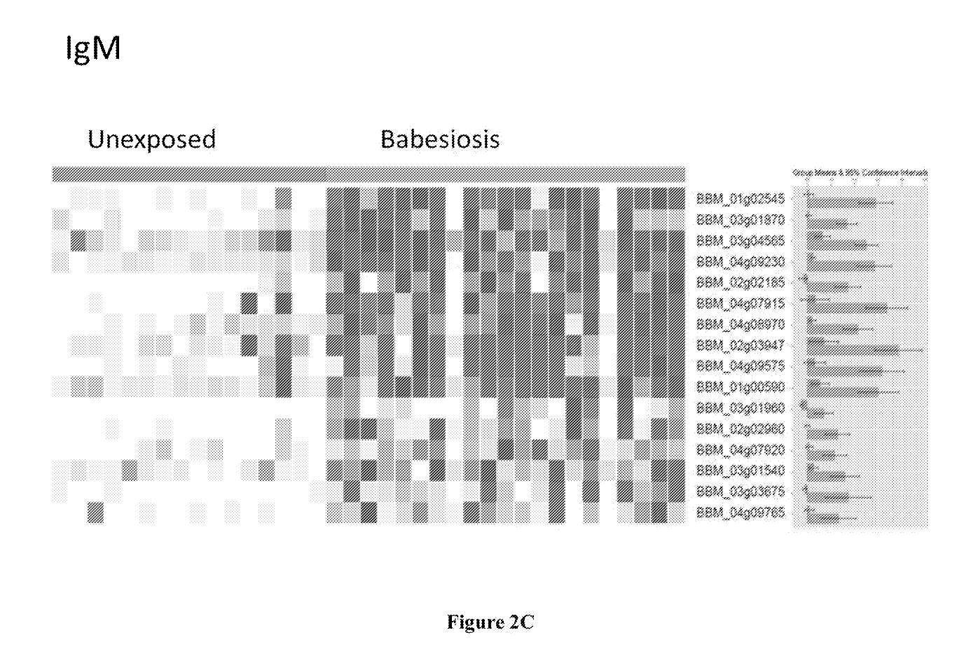

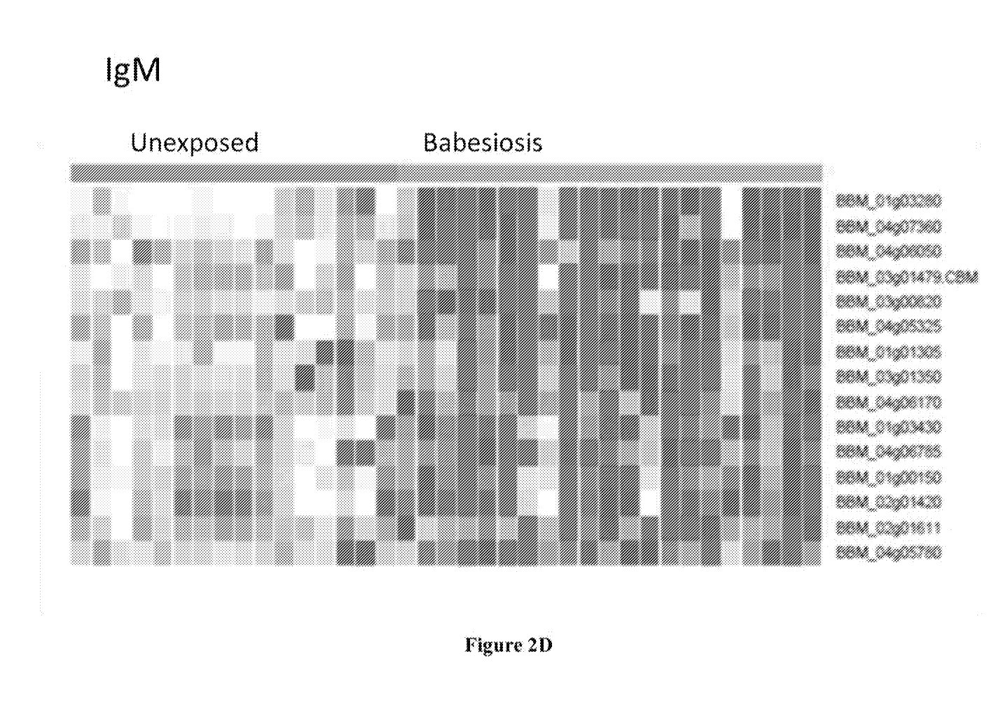

FIGS. 2A-D shows another 16 exemplary antigens selected based on the relative reactivities of antigens to IgG (FIGS. 2A-B) and IgM (FIGS. 2C-D), respectively, represented as heat maps and bar graphs. The heat maps represent the intensity of antibody titer in the sample represented by a color scale (most intensive in black, and least intensive in white). FIG. 2B and FIG. 2D illustrate the heatmap data after scaling signals for each antigen shown in FIG. 2A and FIG. 2C, respectively, so that the antigens with lower reactivity can be discriminatively shown.

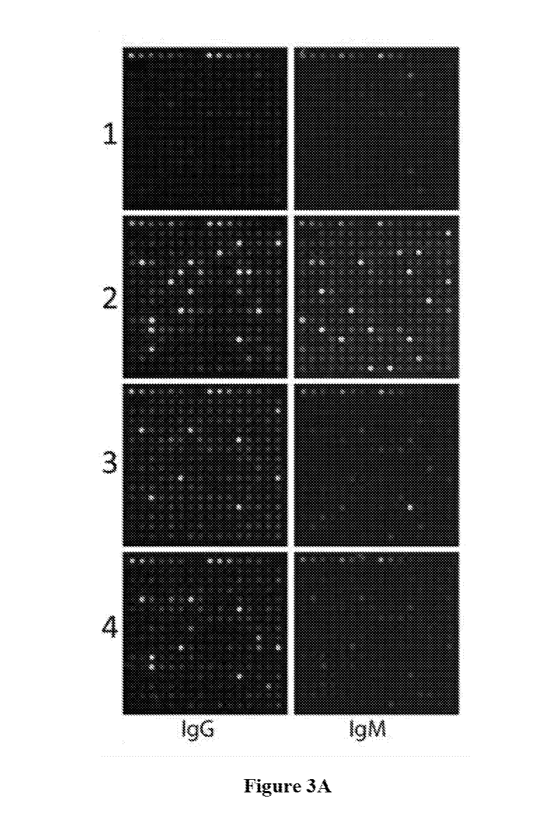

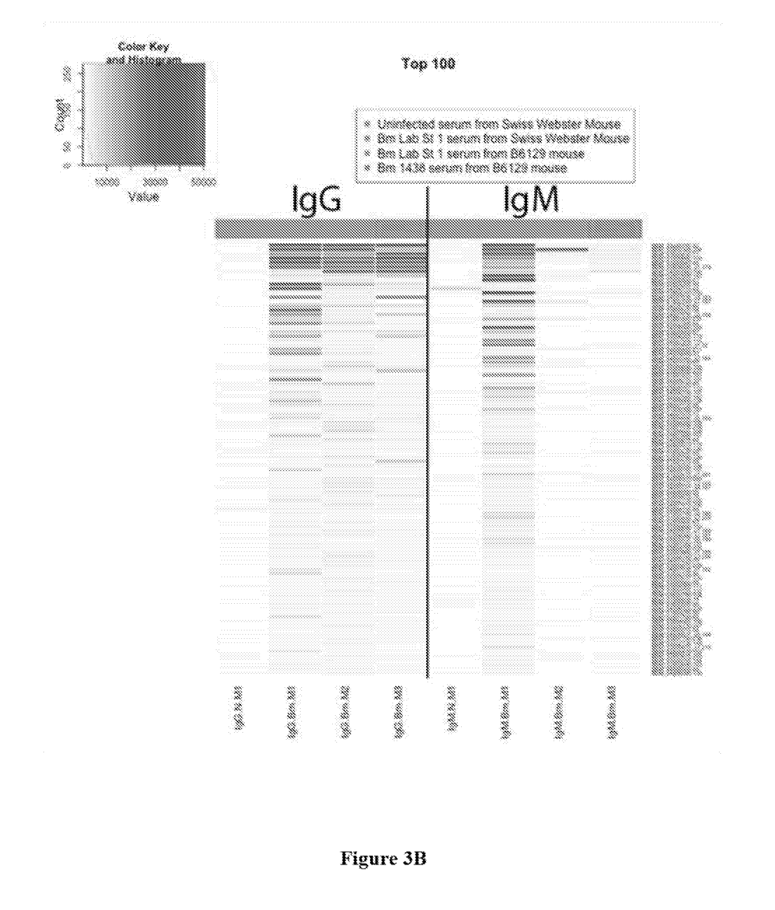

The inventors also tested mouse sera to identify the epitopes that may be conserved because they have little to no evolutionary pressure to change. FIG. 3A shows an exemplary image of B. microti protein arrays that are tested to detect IgG and IgM antibodies in normal serum of uninfected mice and serum of B. microti infected mice. Row 1 shows IgG and IgM test results using uninfected serum from a Swiss Webster mouse. Row 2 shows IgG and IgM test results using infected serum from a Swiss Webster mouse infected with the B. microti LabS1 strain. Row 3 shows IgG and IgM test results using infected serum from a B6129 mouse infected with the B. microti LabS1 strain. Row 4 shows IgG and IgM test results using infected serum from a B6129 mouse infected with the B. microti Bm1438 strain. The test results were again visualized in a heat map shown in FIG. 3B. Thus, it is contemplated that by identification of specific epitopes showing more sensitivity and specificity, a robust and accurate in vitro diagnostic (IVD) test can be created.

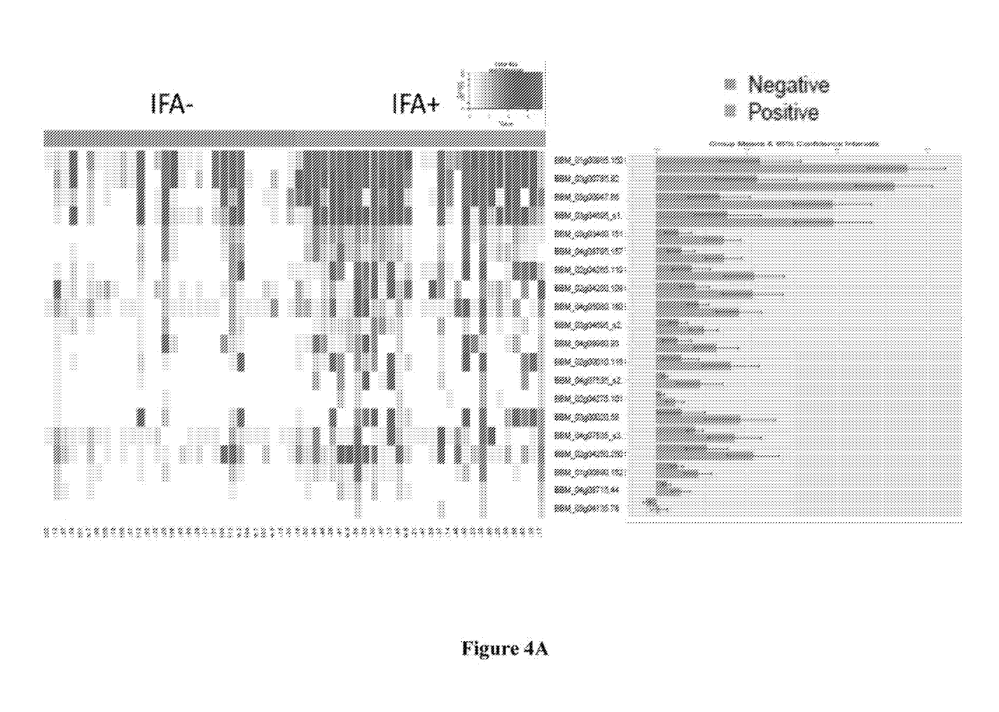

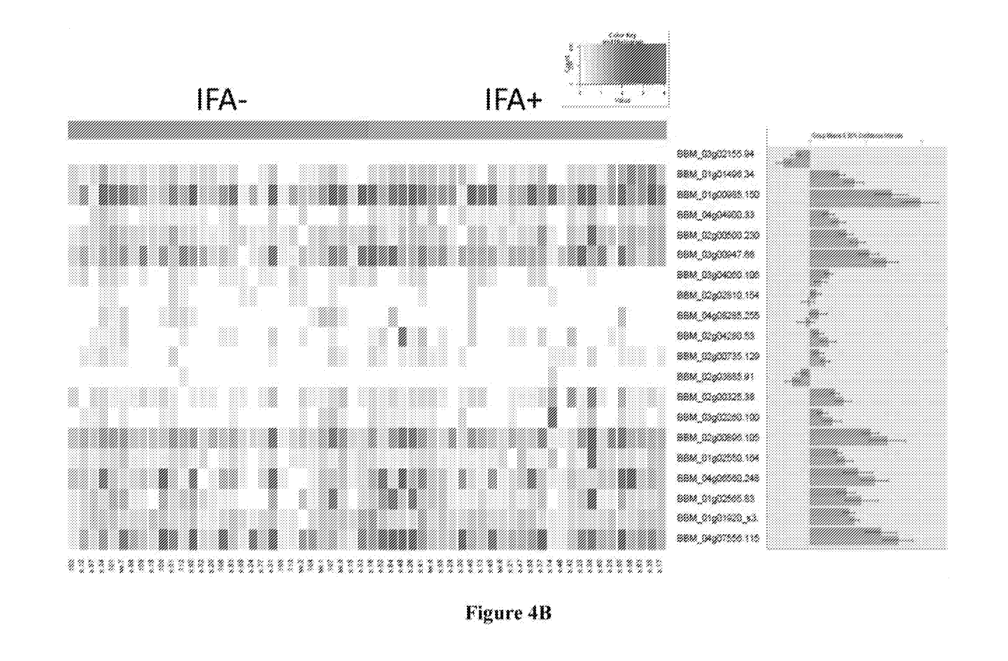

The inventors also tested the IgG and IgM reactivities in wild mice infected with B. microti using B. microti protein arrays used in FIG. 1. The infected wild mice were identified using the IFA “gold standard” test. FIGS. 4A-B shows heat maps and bar graphs representing the top 20 IgG antigens (FIG. 4A) and top 20 IgM antigens (FIG. 4B) identified from the test results using B. microti positive (infected), and negative (uninfected) wild mice sera. The inventors found that top 20 IgG and IgM antigens were similar to the top 20 IgG and IgM antigens identified from infected and uninfected human sera. Furthermore, the inventors found that IgG and IgM antibodies were detected against some of the top proteins in the IFA negative mice. IFA testing relies on the ability to smear infected red blood cells on a slide, and much like the smear testing for other parasites like malaria, has many technical challenges that reduce sensitivity. Sonic of the IFA negative wild mice had detectable levels of IgG and IgM antibodies against several proteins, indicating that a combination of these biomarkers may lead to an improved diagnostic test.

Table 1 shows statistical immunogenicity data of a set of B. microti antigens to human IgG and IgM. Specifically, each antigen is tested using a plurality of human serum infected or uninfected by B. microti. The IgG and IgM antibody titers were measured and mean values of its titers among B. microti infected population (mean positive value) and uninfected population (mean negative value) were presented. Generally, higher positive antibody titer (higher positive value) to an antigen may indicate higher immunogenicity or reactivity of the antigen. To eliminate the false negative or false positive reactivities of some antigens, discriminatory power values were also calculated using antibody titers of infected and uninfected population, including a t-test p-value and an area under the curve (AUC) for each antigen for IgG and IgM. Generally, an at that shows higher discriminatory power values (e.g., t-test p-value<0.1, preferably, t-test p-value<0.05, more preferably, t-test p-value<0.01, an or AUC>0.75, preferably AUC>0.85, more preferably AUC>0.95, etc.) indicates stronger relative reactivities of the antigen. Thus, relative reactivities of antigens can be determined by one or more statistical value indicating discriminatory power values of such antigens.

TABLE 1

| Human IgG | Antigen.ID | P.Value | AUC | Mean.Neg | Mean.Pos | Human IgM |
|---|---|---|---|---|---|---|
| BBM_03g00785.3029 | | 8.99E−10 | 0.95 | 0.399293 | 4.789639 | |
| BBM_03g04695_s1.661 | | 3.05E−09 | 0.93 | 0.669698 | 5.481812 | |
| BBM_01g03280.2214 | | 9.63E−09 | 0.94 | 2.456043 | 6.449811 | |
| BBM_03g00947.3924 | | 8.58E−09 | 0.95 | 0.275067 | 3.86978 | |
| BBM_04g06170.112 | | 0.00239133 | 0.8 | 1.196694 | 2.81381 | |
| BBM_04g07360.2433 | | 6.77E−11 | 0.94 | 0.755438 | 5.648211 | |
| BBM_01g01305.3364 | | 0.00022647 | 0.84 | 2.68284 | 4.747776 | |
| BBM_03g01350.3631 | | 1.33E−05 | 0.85 | 1.046079 | 3.35897 | |
| BBM_01g03430.1210 | | 0.8555205 | 0.46 | 5.223108 | 5.140795 | |
| BBM_01g00150.2961 | | 0.9056553 | 0.47 | 4.576688 | 4.524298 | |
| BBM_04g06785.4411 | | 0.01999478 | 0.72 | 2.77311 | 4.087223 | |
| BBM_03g00820.2139 | | 5.81E−07 | 0.9 | 1.587474 | 4.719381 | |
| BBM_04g05780.3646 | | 0.34480926 | 0.64 | 2.238249 | 2.70729 | |
| BBM_02g01420.4098 | | 0.89471116 | 0.48 | 4.015039 | 3.954798 | |
| BBM_02g01611.3913 | | 7.86E−03 | 0.9 | −0.3352 | 0.78502 | |
| BBM_04g09840.4118 | | 0.90989476 | 0.47 | 4.460904 | 4.409516 | |
| BBM_03g00020.2501 | | 0.00035712 | 0.87 | 0.496188 | 2.351402 | |
| BBM_01g01620_s1.2140 | | 0.00183958 | 0.77 | 1.598854 | 2.947658 | |
| BBM_03g00840.3233 | | 0.87324552 | 0.46 | 3.98483 | 3.914705 | |
| BBM_03g03976.3506 | | 0.92943889 | 0.48 | 4.802056 | 4.761534 | |
| BBM_01g00590.384 | | 2.02E−05 | 0.91 | 0.541924 | 3.003248 | |
| BBM_01g00500.3846 | | 0.08659995 | 0.68 | 1.178904 | 2.111308 | |
| BBM_02g02170.2365 | | 0.91005807 | 0.48 | 4.892041 | 4.840592 | |
| BBM_02g00910.2993 | | 0.37235784 | 0.46 | 2.349886 | 1.815883 | |
| BBM_03g00452.1539 | | 0.00334588 | 0.88 | 0.114319 | 0.927211 | |
| BBM_01g01135.421 | | 0.04666509 | 0.68 | 1.805466 | 2.996336 | |
| BBM_04g09615_e1of2_s1of1.3781 | | 0.09067509 | 0.67 | 1.550336 | 2.364191 | |
| BBM_01g00985_s2.2509 | | 1.84E−06 | 0.9 | 0.842178 | 3.922386 | |
| BBM_01g00985_s1.3665 | | 7.70E−07 | 0.9 | 1.272292 | 4.523013 | |
| BBM_03g00450.1002 | | 0.9485982 | 0.58 | 1.840978 | 1.811501 | |
| BBM_03g04295.771 | | 0.03438416 | 0.76 | 0.023603 | 0.364221 | |
| BBM_01g00435.596 | | 0.72592556 | 0.56 | 1.679258 | 1.841831 | |
| BBM_03g04505.2457 | | 0.07771156 | 0.67 | 0.781672 | 1.217634 | |
| BBM_01g00160.2425 | | 0.00555469 | 0.77 | 0.445386 | 1.476276 | |
| BBM_03g01075_s2.4543 | | 0.01491108 | 0.69 | 0.471006 | 0.992889 | |
| BBM_02g04265.3564 | | 0.01011674 | 0.78 | 0.913018 | 2.288798 | |
| BBM_03g02305.2730 | | 0.00090101 | 0.8 | 0.76321 | 2.316844 | |
| BBM_03g03046.750 | | 0.00389917 | 0.75 | 1.790389 | 3.603164 | |
| BBM_03g01622_e1of3_s1of1.1464 | | 0.00225706 | 0.84 | 0.58517 | 1.82656 | |
| BBM_02g00975.2145 | | 0.00567154 | 0.77 | 0.318504 | 1.091411 | |
| BBM_02g00810_s2.800 | | 0.00471435 | 0.77 | 0.11524 | 1.147255 | |
| BBM_04g08570.3912 | | 0.4258462 | 0.67 | 0.514197 | 0.725369 | |
| BBM_03g01705_e1of5_s1of1.4356 | | 0.00031077 | 0.84 | 0.376543 | 1.425722 | |
| BBM_04g07915_s1.3376 | | 0.10883313 | 0.65 | 2.230289 | 3.294055 | |
| BBM_01g00985.2215 | | 2.70E−07 | 0.92 | 1.212289 | 4.288104 | |
| BBM_03g00960.4212 | | 8.38E−09 | 0.93 | 0.460178 | 2.947206 | |
| BBM_03g03925.1879 | | 0.00011368 | 0.84 | 0.264361 | 1.584464 | |
| BBM_02g01875.2757 | | 0.02031508 | 0.7 | 1.270701 | 2.372419 | |
| BBM_04g05680.1865 | | 0.00104163 | 0.79 | 0.434877 | 1.578809 | |
| BBM_04g09295.3890 | | 0.62386187 | 0.62 | 0.64532 | 0.8143 | |
| BBM_02g01015_e1of6_s1of1.4036 | | 0.015301 | 0.82 | 0.12908 | 0.484692 | |
| BBM_03g02515_s1.431 | | 0.0086695 | 0.71 | 1.129206 | 2.229231 | |
| BBM_03g00315_s2.3982 | | 0.02945243 | 0.73 | 0.884862 | 1.799952 | |
| BBM_04g05260_s1.2831 | | 0.03992059 | 0.7 | 2.035397 | 3.124443 | |
| BBM_03g02830.468 | | 0.00070694 | 0.84 | 0.314615 | 1.432756 | |
| BBM_01g01800.1195 | | 0.09548049 | 0.62 | 0.944946 | 1.616112 | |
| BBM_02g04285.2351 | | 3.23E−07 | 0.91 | 0.621857 | 4.25867 | |

| Human IgG | P.Value | AUC | Mean.Neg | Mean.Pos |
|---|---|---|---|---|
| BBM_03g00785.3029 | 4.99E−07 | 0.940476 | 0.095599 | 2.891633 |
| BBM_03g04695_s1.661 | 4.11E−06 | 0.910714 | 0.272967 | 2.256857 |
| BBM_01g03280.2214 | 9.43E−08 | 0.922619 | 0.664363 | 2.553439 |
| BBM_03g00947.3924 | 3.34E−05 | 0.91369 | 0.326227 | 2.165435 |
| BBM_04g06170.112 | 0.000245 | 0.925595 | 0.247496 | 2.047512 |
| BBM_04g07360.2433 | 8.43E−07 | 0.916667 | 0.209352 | 2.006523 |
| BBM_01g01305.3364 | 0.000203 | 0.866071 | 0.497504 | 2.098612 |
| BBM_03g01350.3631 | 0.00024 | 0.854167 | 0.403865 | 1.950929 |
| BBM_01g03430.1210 | 0.000344 | 0.842262 | 1.453502 | 2.821564 |
| BBM_01g00150.2961 | 0.000386 | 0.842262 | 0.744639 | 2.093507 |
| BBM_04g06785.4411 | 0.000349 | 0.848214 | 1.190192 | 2.520046 |
| BBM_03g00820.2139 | 2.95E−05 | 0.863095 | 0.444266 | 1.730086 |
| BBM_04g05780.3646 | 0.001536 | 0.869048 | 0.432923 | 1.683829 |
| BBM_02g01420.4098 | 0.00068 | 0.824405 | 0.714072 | 1.816276 |
| BBM_02g01611.3913 | 0.001085 | 0.83631 | −0.30714 | 0.778514 |
| BBM_04g09840.4118 | 0.001457 | 0.794643 | 1.038822 | 2.112978 |
| BBM_03g00020.2501 | 0.004288 | 0.83631 | 0.186958 | 1.206587 |
| BBM_01g01620_s1.2140 | 0.004634 | 0.770833 | 0.772469 | 1.782977 |
| BBM_03g00840.3233 | 0.001287 | 0.797619 | 0.702094 | 1.705535 |
| BBM_03g03976.3506 | 0.002931 | 0.732143 | 1.300336 | 2.295758 |
| BBM_01g00590.384 | 0.000807 | 0.895833 | 0.24302 | 1.209739 |
| BBM_01g00500.3846 | 0.002314 | 0.72619 | 0.30527 | 1.224276 |
| BBM_02g02170.2365 | 0.003017 | 0.738095 | 1.27648 | 2.17413 |
| BBM_02g00910.2993 | 0.000295 | 0.830357 | 0.183629 | 1.079076 |
| BBM_03g00452.1539 | 0.000242 | 0.889881 | −0.01193 | 0.880743 |
| BBM_01g01135.421 | 0.007734 | 0.848214 | 0.337546 | 1.21898 |
| BBM_04g09615_e1of2_s1of1.3781 | 0.001632 | 0.821429 | 0.471247 | 1.344285 |
| BBM_01g00985_s2.2509 | 0.000443 | 0.842262 | 0.119733 | 0.988987 |
| BBM_01g00985_s1.3665 | 0.000489 | 0.827381 | 0.601772 | 1.45991 |
| BBM_03g00450.1002 | 0.000435 | 0.821429 | 0.418348 | 1.269846 |
| BBM_03g04295.771 | 0.004563 | 0.869048 | 0.001928 | 0.834462 |
| BBM_01g00435.596 | 0.000649 | 0.860119 | 0.412148 | 1.229173 |
| BBM_03g04505.2457 | 0.003029 | 0.827381 | 0.302568 | 1.11793 |
| BBM_01g00160.2425 | 0.001725 | 0.833333 | 0.173807 | 0.979739 |
| BBM_03g01075_s2.4543 | 0.012213 | 0.809524 | 0.205262 | 1.007971 |
| BBM_02g04265.3564 | 0.00104 | 0.815476 | 0.092835 | 0.893672 |
| BBM_03g02305.2730 | 0.013936 | 0.747024 | 0.640078 | 1.433619 |
| BBM_03g03046.750 | 0.000249 | 0.877976 | 0.130485 | 0.911207 |
| BBM_03g01622_e1of3_s1of1.1464 | 0.005429 | 0.779762 | 0.360475 | 1.138501 |
| BBM_02g00975.2145 | 0.006667 | 0.824405 | −0.01446 | 0.757339 |
| BBM_02g00810_s2.800 | 0.00166 | 0.848214 | 0.136859 | 0.905849 |
| BBM_04g08570.3912 | 0.000561 | 0.860119 | 0.024639 | 0.787656 |
| BBM_03g01705_e1of5_s1of1.4356 | 0.002535 | 0.872024 | 0.233708 | 0.995128 |
| BBM_04g07915_s1.3376 | 0.000111 | 0.883929 | 0.154629 | 0.908279 |
| BBM_01g00985.2215 | 0.002099 | 0.785714 | 0.426908 | 1.176901 |
| BBM_03g00960.4212 | 0.000121 | 0.860119 | 0.078647 | 0.822714 |
| BBM_03g03925.1879 | 0.008902 | 0.803571 | −0.03924 | 0.702946 |
| BBM_02g01875.2757 | 0.007509 | 0.764881 | 0.02689 | 0.76859 |
| BBM_04g05680.1865 | 0.003561 | 0.764881 | 0.031809 | 0.751091 |
| BBM_04g09295.3890 | 0.006017 | 0.758929 | 0.036413 | 0.754353 |
| BBM_02g01015_e1of6_s1of1.4036 | 0.011264 | 0.791667 | −0.07767 | 0.635066 |
| BBM_03g02515_s1.431 | 0.036854 | 0.669643 | 0.472547 | 1.182825 |
| BBM_03g00315_s2.3982 | 0.000476 | 0.815476 | 0.078946 | 0.788293 |
| BBM_04g05260_s1.2831 | 0.00137 | 0.776786 | 0.257747 | 0.966254 |
| BBM_03g02830.468 | 0.008461 | 0.839286 | 0.125011 | 0.828582 |
| BBM_01g01800.1195 | 0.005549 | 0.782738 | 0.243352 | 0.942951 |
| BBM_02g04285.2351 | 0.008494 | 0.797619 | 0.245089 | 0.944029 |

IgG and IgM antigens can be ranked according to its relative reactivities. Generally, an antigen with higher mean positive value, lower mean negative value, and lower t-test p-value (and/or higher AUC value) would be, ranked higher as such antigen is predicted to be highly specific to only infected population and show stronger signal (higher antibody titer) in the test. Table 2 illustrates an exemplary group of top candidate antigens that can be used as biomarkers for B. microti infection or onset/progress of Babesiosis. Each antigen in this exemplary group is ranked based on its specificity and sensitivity to IgG and IgM reactivity data.

TABLE 2

| Description | Gene.ID | ID | IgG.Rank | IgM.Rank | Total Rank |
|---|---|---|---|---|---|
| BMG09; BMN1-9; BmSA1 | BBM_03g00785 | BBM_03g00785 | 1 | 1 | 1 |
| rhoptry neck protein 2 | BBM_03g04695 | BBM_03g04695_s1 | 2 | 2.5 | 2 |
| hypothetical protein | BBM_01g00985 | BBM_01g00985 | 3 | 16.5 | 3 |
| hypothetical protein | BBM_03g00947 | BBM_03g00947 | 4 | 2.5 | 4 |
| hypothetical protein | BBM_03g00960 | BBM_03g00960 | 5 | 5 | 5 |
| rhoptry neck protein 2 | BBM_03g04695 | BBM_03g04695_s2 | 6 | 20.5 | 6 |
| Nucleoporin FG repeat region | BBM_03g02345 | BBM_03g02345 | 7 | 25.5 | 7 |
| BMN family (2) | BBM_04g09980 | BBM_04g09980 | 8 | 25.5 | 8 |
| DNA mismatch repair protein | BBM_03g03480 | BBM_03g03480 | 10 | 35.5 | 9 |
| hypothetical protein | BBM_04g06560 | BBM_04g06560 | 10 | 31 | 10 |
| hypothetical protein | BBM_02g04285 | BBM_02g04285 | 10 | 33 | 11 |
| Actin | BBM_03g02390 | BBM_03g02390 | 12 | 23 | 12 |
| BMN family (2) | BBM_02g04265 | BBM_02g04265 | 14 | 6 | 13 |
| hypothetical protein | BBM_04g05080 | BBM_04g05080 | 14 | 38 | 14 |
| BMN family-2 | BBM_03g00020 | BBM_03g00020 | 14 | 14 | 15 |
| hypothetical protein | BBM_02g02608 | BBM_02g02608 | 16 | 35.5 | 16 |
| BMN family (2), MN-10 | BBM_02g04280 | BBM_02g04280 | 17 | 29.5 | 17 |
| BMG08; BMN family-1 | BBM_02g04260 | BBM_02g04260 | 18 | 29.5 | 18 |
| S1/P1; Nuclease; PredGPI | BBM_02g03140 | BBM_02g03140 | 9.5 | | 19 |
| BMG14 | BBM_02g00896 | BBM_02g00896 | 20 | 24 | 20 |
| BMG20 | BBM_03g03430 | BBM_03g03430 | 21 | 4 | 21 |
| BMN family (2) | BBM_02g00010 | BBM_02g00010 | 22.5 | 16.5 | 22 |
| BMG07, N1-21a orthologue | BBM_02g04275 | BBM_02g04275 | 22.5 | 37 | 23 |
| BMG10, BMN family, CDS | BBM_03g00790 | BBM_03g00790 | 24.5 | 28 | 24 |
| hypothetical protein | BBM_02g02940 | BBM_02g02940 | 24.5 | 7 | 25 |
| hypothetical protein, No PredGBBPMI | BBM_04g07556 | BBM_04g07556 | 26 | 27 | 26 |
| BMG13, Sexual stage antigen, | BBM_04g07810 | BBM_04g07810 | 27 | 34 | 27 |
| Protein dopey-1 | BBM_02g04060 | BBM_02g04060_s2 | 28 | 8 | 28 |
| hypothetical protein | BBM_04g06750 | BBM_04g06750 | 29.5 | 19 | 29 |
| hypothetical protein | BBM_01g01510 | BBM_01g01510 | 29.5 | 22 | 30 |
| leucyl aminopeptidase | BBM_02g03960 | BBM_02g03960 | 31 | 39 | 31 |
| structural maintenance of | BBM_01g02740 | BBM_01g02740_s2 | 32.5 | 11 | 32 |
| hypothetical protein | BBM_02g04250 | BBM_02g04250 | 32.5 | 32 | 33 |
| structural maintenance of | BBM_01g02740 | BBM_01g02740_s1 | 34 | 13 | 34 |
| Vacuolar protein sorting-assoc | BBM_04g05345 | BBM_04g05345_s2 | 35 | 12 | 35 |
| BMG12, BLAST homologies | BBM_03g02840 | BBM_03g02840 | 36 | 16.5 | 36 |
| BMN family (1), pseudo gene | BBM_02g00005 | BBM_02g00005 | 37 | 9.5 | 37 |
| hypothetical protein | BBM_01g02415 | BBM_01g02415_s1 | 38 | 20.5 | 38 |
| hypothetical protein | BBM_03g04760 | BBM_03g04760 | 39 | 16.5 | 39 |

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An antigen composition comprising:
a plurality of antibody reactive antigens associated with a carrier, wherein at least two of the antigens have quantified and known relative antibody reactivities with respect to sera of a population infected by Babesia microti; and
wherein the at least one of the plurality of antigens are selected from the group consisting of BBM_03g00785, BBM_03g04695, BBM_01g00985, BBM_03g00947, BBM_03g00960, BBM_03g04695, BBM_03g02345, BBM_04g09980, BBM_03g03480, BBM_04g06560, BBM_02g04285, BBM_03g02390, BBM_02g04265, BBM_04g05080, BBM_03g00020, BBM_02g02608, BBM_02g04280, BBM_02g04260, BBM_02g03140, BBM_02g00896, BBM_03g03430, BBM_02g00010, BBM_02g04275, BBM_03g00790, BBM_02g02940, BBM_04g07556, BBM_04g07810, BBM_02g04060, BBM_04g06750, BBM_01g01510, BBM_02g03960, BBM_01g02740, BBM_02g04250, BBM_01g02740, BBM_04g05345, BBM_03g02840, BBM_02g00005, BBM_01g02415, BBM_03g04760, BBM_01g02545, BBM_03g01870, BBM_03g04565, BBM_04g09230, BBM_02g02185, BBM_04g07915, BBM_04g08970, BBM_02g03947, BBM_04g09575, BBM_01g00590, BBM_03g01960, BBM_02g02960. BBM_04g07920, BBM_03g01540, BBM_03g03675, BBM_04g09765, or fragments thereof.

2. The antigen composition of claim 1, wherein the known reactivities are characterized by strength of reactivity.

3. The antigen composition of claim 1, wherein the at least one of the plurality of antigens have an average discriminatory p value of ≤0.05.

4. The antigen composition of claim 1, wherein the known relative antibody reactivities are IgG reactivities or IgM reactivities

5. (canceled)

6. The antigen composition of claim 1, wherein the known relative antibody reactivities are at least 30% higher compared to with respect to sera of a population uninfected by Babesia microti.

7. (canceled)

8. The antigen composition of claim 1, wherein the carrier is a solid carrier, and the plurality of antigens is disposed on the solid carrier in an array.

9. (canceled)

10. The antigen composition of claim 1, wherein antibodies to the at least two antigens are present in at least 60% of a population infected by Babesia microti.

11. A method of predicting a likelihood of an individual having Babesia microti infection, comprising:
providing a serum sample of the individual;
reacting the serum sample with a plurality of antigens antibody reactive antigens associated with a carrier, wherein at least two of the antigens have quantified and known relative antibody reactivities with respect to sera of a population infected by Babesia microti;
wherein the at least one of the plurality of antigens are selected from the group consisting of BBM_03g00785, BBM_03g04695, BBM_01g00985, BBM_03g00947, BBM_03g00960, BBM_03g04695, BBM_03g02345, BBM_04g09980, BBM_03g03480, BBM_04g06560, BBM_02g04285, BBM_03g02390, BBM_02g04265, BBM_04g05080, BBM_03g00020, BBM_02g02608, BBM_02g04280, BBM_02g04260, BBM_02g03140, BBM_02g00896, BBM_03g03430, BBM_02g00010, BBM_02g04275, BBM_03g00790, BBM_02g02940, BBM_04g07556, BBM_04g07810, BBM_02g04060, BBM_04g06750, BBM_01g01510, BBM_02g03960, BBM_01g02740, BBM_02g04250, BBM_01g02740, BBM_04g05345, BBM_03g02840, BBM_02g00005, BBM_01g02415, BBM_03g04760, BBM_01g02545, BBM_03g01870, BBM_03g04565, BBM_04g09230, BBM_02g02185, BBM_04g07915, BBM_04g08970, BBM_02g03947, BBM_04g09575, BBM_01g00590, BBM_03g01960, BBM_02g02960. BBM_04g07920, BBM_03g01540, BBM_03g03675, BBM_04g09765, or fragments thereof.

12. The method of claim 11, wherein the plurality of the antigens have a known association with a disease parameter, wherein the disease parameter is selected from the group consisting of a previous or current exposure to a pathogen, acute infection with a pathogen, latent or recurrent infection with a pathogen, and at least partial immunity to infection with a pathogen.

13. The method of claim 11, wherein the carrier is a pharmaceutically acceptable carrier, and wherein the composition is formulated as a vaccine.

14. The method of claim 11, wherein the carrier is a solid carrier, and the plurality of antigens is disposed on the carrier in an array.

15. The method of claim 11, wherein at least one of the plurality of antigens or fragments thereof are recombinant.

16. The method of claim 11, wherein the plurality of antigens or fragments thereof are at least partially purified.

17. (canceled)

18. (canceled)

19. (canceled)

20. (canceled)

21. (canceled)

22. A diagnostic kit for testing Babesia microti infection in patients suspected to have Babesia microti infection, comprising:
a plurality of antibody reactive antigens associated with a carrier, wherein at least two of the antigens have quantified and known relative antibody reactivities with respect to sera of a population infected by Babesia microti; and
wherein the at least one of the plurality of antigens are selected from the group consisting of BBM_03g00785, BBM_03g04695, BBM_01g00985, BBM_03g00947, BBM_03g00960, BBM_03g04695, BBM_03g02345, BBM_04g09980, BBM_03g03480, BBM_04g06560, BBM_02g04285, BBM_03g02390, BBM_02g04265, BBM_04g05080, BBM_03g00020, BBM_02g02608, BBM_02g04280, BBM_02g04260, BBM_02g03140, BBM_02g00896, BBM_03g03430, BBM_02g00010, BBM_02g04275, BBM_03g00790, BBM_02g02940, BBM_04g07556, BBM_04g07810, BBM_02g04060, BBM_04g06750, BBM_01g01510, BBM_02g03960, BBM_01g02740, BBM_02g04250, BBM_01g02740, BBM_04g05345, BBM_03g02840, BBM_02g00005, BBM_01g02415, BBM_03g04760, BBM_01g02545, BBM_03g01870, BBM_03g04565, BBM_04g09230, BBM_02g02185, BBM_04g07915, BBM_04g08970, BBM_02g03947, BBM_04g09575, BBM_01g00590, BBM_03g01960, BBM_02g02960. BBM_04g07920, BBM_03g01540, BBM_03g03675, BBM_04g09765, or fragments thereof.

23. The test kit of claim 22, wherein the known reactivities are characterized by strength of reactivity.

24. The test kit of claim 22, wherein the at least one of the plurality of antigens have an average discriminatory p value of≤0.05.

25. The test kit of claim 22, wherein the known relative antibody reactivities are IgG reactivities or IgM reactivities

26. (canceled)

27. The test kit of claim 22, wherein the known relative antibody reactivities are at least 30% higher compared to with respect to sera of a population uninfected by Babesia microti.

28. (canceled)

29. The test kit of claim 22, wherein the carrier is a solid carrier, and the plurality of antigens is disposed on the solid carrier in an array.

30. (canceled)

31. The test kit of claim 22, wherein antibodies to the at least two antigens are present in at least 60% of a population infected by Babesia microti.
